(19) United States
(12) Patent Application Publication
(10) Pub. No.: US 2024/0278234 A1
(43) Pub. Date: Aug. 22, 2024

(54) SAMPLE PREPARATION

(71) Applicant: Readygo Diagnostics Limited, North Wraxall, Chippenham (GB)

(72) Inventor: Benjamin David Cobb, North Wraxall (GB)

(21) Appl. No.: 18/571,723

(22) PCT Filed: Jun. 20, 2022
(86) PCT No.: PCT/EP2022/066737

§ 371 Date: Dec. 19, 2023

(30) Foreign Application Priority Data
Jun. 24, 2021 (GB) 2109050.1
Jul. 23, 2021 (GB) 2110617.4

(51) Int. Cl. B01L 3/00; A61B 10/00; B01L 3/02; C12N 15/10; G01N 1/40

(52) U.S. Cl. CPC B01L 3/5023; A61B 10/0051; B01L 3/0275; C12N 15/1003; G01N 1/405; B01L 2300/0681; B01L 2300/069; B01L 2300/0832

(57) ABSTRACT

Described is a disposable device for collection and processing of biological samples, particularly for the purpose of DNA and RNA analysis procedures. The device includes (a) a hollow tip comprising first and second generally cylindrical sections and having first and second openings at first and second ends of the tip, wherein the second section and second opening have a smaller diameter than the first section and first opening, and wherein the first section and first opening are configured to removably attach to a sampling instrument; and (b) a porous nib having a working surface at a first end exposed or exposable for acquiring a biological sample, a porous structure suitable for the absorption of biological sample matter thus acquired, and for the passage of liquid through the nib; and a washing surface at a second end for application of a wash fluid to the nib. Also described is the use of polyvinylpyrrolidone, chlorhexidine, or citral as functionalising agents within the porous nib to aid release of nucleic acids without inhibiting subsequent sample processing.

### FIELD OF THE INVENTION

The present invention relates to collecting clinical samples and preparing them for analysis, for example by molecular biology processing techniques such as nucleic acid amplification and/or detection; and more particularly to a sampling device for that purpose, especially useful in a clinical point of care (POC) or point of need (PON) setting, but also for use in sending to a remote laboratory for testing. Certain aspects of the invention relate to processes and compositions useful in preparation of samples for nucleic acid amplification and/or detection.

### BACKGROUND TO THE INVENTION

The Polymerase Chain Reaction (PCR) is a convenient method of amplifying nucleic acids, employed to test for the presence of specific nucleic acids (DNA or RNA) in a biological sample. It is used among other purposes as a diagnostic method to identify markers for pathogens or diseases. Other methods of amplifying nucleic acid samples include isothermal amplification methods such as Recombinase Polymerase Amplification (RPA) and Loop-Mediated Isothermal Amplification (LAMP).

Prior to any such diagnostic test there needs to be a certain amount of preparation of the sample in order to present nucleic acids in a state that is compatible with the amplification process being used. Laboratory-based extraction methods are generally geared towards providing high concentrations of high purity nucleic acid, the aim being to obtain as much nucleic acid as possible. This comes at the expense of simplicity, and lab-based extraction techniques often require access to reagents and equipment such as centrifuges that are not compatible with clinical settings.

Preparation of clinical samples requires just enough nucleic acid to perform the test. This is an important distinction because it offers significant simplification of the extraction process. It is known for example, that the use of a simple filter paper enables the capture of enough nucleic acid from a target in a biological sample for a subsequent diagnostic via PCR, even from complex sample matrices such as whole blood (Fuehrer et al. J Clin Microbiol. 2011, 49(4), 1628-1630; Bu et al. Analytical Biochemistry 375(2), 370-372; Zou et al (2017) Nucleic acid purification from plants, animals and microbes in under 30 seconds, PLoS Biol 15(11), e2003916). However, one problem can be that certain reagents which may be useful in preparation of samples can interfere with downstream techniques such as PCR amplification.

One way in which a biological sample may be taken involves the use of a porous nib which can be retained within a sampling device, which can resemble for example a pen. One such device is described in US Patent Application Publication US 2016/0349153, which describes a device having a housing containing a sintered porous matrix in the form of a porous nib which can absorb a liquid sample through capillary action. A different device is described in our international patent application PCT/EP2021/057315, which includes a reservoir for containing a buffer liquid for flushing adsorbed sample from the nib for subsequent processing and analysis.

### SUMMARY OF THE INVENTION

Described here is a disposable device for collection and processing of biological samples, particularly—but not exclusively—for the purpose of DNA and RNA analysis procedures. The device provides enough nucleic acid for a molecular diagnostic test. Although the device is primarily described herein in conjunction with use in molecular diagnostic testing for nucleic acids, it will be appreciated that other biomolecules may be collected and/or processed with the invention, for example, proteins, lipids, metabolites, vitamins, drugs, organics, pesticide, small molecules, etc; as may cell fragments such as cell membrane or cell coat fragments, or subcellular organelles. We have determined that a particular nib configuration allows for effective release of sample from the nib without the need to actively flush a buffer liquid through the nib by applying pressure.

According to a first aspect of the present invention there is provided a device for obtaining and optionally processing biological samples for analysis, comprising:
(a) a hollow tip comprising first and second generally cylindrical sections and having first and second openings at first and second ends of the tip, wherein the second section and second opening have a smaller diameter than the first section and first opening, and wherein the first section and first opening are configured to removably attach to a sampling instrument; and
(b) a porous nib having a working surface at a first end exposed or exposable for acquiring a biological sample, a porous structure suitable for the absorption of biological sample matter thus acquired, and for the passage of liquid through the nib; and a washing surface at a second end for application of a wash fluid to the nib; and
wherein the second section and second opening are configured to removably receive the nib, and wherein the porous nib has a length which is greater than the width.

We have found that, surprisingly, the configuration of this tip and nib not only allows sample to be absorbed into the porous structure through the working surface, but also allows wash fluid to be applied to the washing surface and thereby elute sample from the nib—for example, for subsequent processing or analysis—purely by capillary action and gravity. It is not necessary for pressure to be applied to actively push the wash fluid through the nib to elute the sample. Further, the manner in which the device operates means that there is reduced risk of generating aerosols from samples—which may potentially contain pathogens. As a sample is first absorbed into the nib, and later washed out, by capillary action and gravity, it is not necessary to vigorously pipette the sample, which process can generate aerosols.

Note that the length and width of the nib can be defined by the structure of the nib and tip. In general, the nib has a first end which defines the working surface and a second end which defines the washing surface. The axis extending between these first and second ends defines the length of the nib, while the width is perpendicular to this axis. Where the nib is generally cylindrical, the width may be the diameter of the cylinder; although in some embodiments the nib need not have a constant width; here the part of greatest width is smaller than the length.

When the nib is received within the second section and the second opening, the working surface of the nib is exposed, and the washing surface is within the tip. Preferably the washing surface is within the tip at a level with the transition between first and second sections of the tip; that is, the tip widens—in the form of the first section—above the washing surface of the nib.

The height of the tip above the nib allows a ‘head’ of wash fluid sufficient to break the surface tension of the liquid held in the nib and allow flow of material under gravity. This can be modulated using viscosity and surface tension modifiers in the wash fluid. Preferably the length of the first section of the tip is greater than the diameter of the same section; and preferably also greater than the length of the nib. Unexpectedly, the capillarity of the nib required in order to absorb sample would be expected to saturate the nib and retain the sample without using pressure to force the wash fluid through the nib. However, we have found that the configuration allows almost the same volume of liquid to drop through the saturated material releasing extracted nucleic acids and proteins from sample material that was soaked into it. The efficiency of release is unexpected.

The use of the hollow tip structure allows the tip and nib to be attached to a sampling instrument. Conveniently, this may be a pipette or similar handling device. For example, multitip and/or robotic pipettes may be useful for rapid handling and processing of multiple samples. Alternatively, the sampling instrument may simply be a handle which engages with the tip to allow for handling and manipulation.

In use, the tip and nib may be envisioned as operating as follows. A user attaches the tip and nib to a pipette. The nib is then placed in a patient's mouth, and allowed to absorb saliva which will include nucleic acids for testing. The user then holds the pipette over a test tube or sample plate, and ejects the tip from the pipette so as to release the tip and nib into the test tube. The same pipette can then be used to add wash fluid to the upper surface of the nib within the tip. Under gravity and/or capillary action, the wash fluid then passes into the nib and elutes the sample from the lower surface of the nib into the test tube. The nib and tip can then be removed, and the eluted sample analysed using any suitable procedure. In some embodiments, the test tube or sample plate may contain a bibulous member or bibulous paper which in turn will adsorb any eluted sample. In other embodiments, a saliva sample may first be collected in a test tube or other container. The tip/nib combination can then collect saliva sample from the test tube, in much the same way as directly from the mouth. In a still further variant, the nib alone can be placed in the test tube with the saliva sample. The nib will then absorb saliva, and the user can then place the nib within a tip which is located on a pipette, and proceed to further processing in a similar manner.

The present invention provides a convenient means of obtaining and preparing a sample for analysis, particularly by nucleic acid amplification processes such as PCR, RPA or LAMP, but also potentially by other analytic approaches such as immunodetection. This is achieved, at least in part, by the nib that is used to collect the sample by absorption of liquid or part-liquid, or to wipe a surface containing the biological sample.

The nib may be of generally cylindrical form, although any suitable shape may be used, provided the nib is longer than it is wide. For example, a generally cylindrical nib may include curved or rounded faces at either or both of the working and washing surfaces; or the angle formed between the working and/or washing surface and the body of the nib may be greater than 90 degrees. These configurations can improve patient comfort while sample collection is being carried out, and may also reduce edge effects on capillary holding which may otherwise be seen with a strictly cylindrical nib. In one form, the nib has a chisel- or slanted chisel-shaped working surface to facilitate acquisition of sample at a fine point, at a fine edge, or as a broad stroke. This may be particularly useful in forensics for obtaining samples from various surfaces.

The nib may be held within the tip by an interference fit; for example, one or more annular rings may be formed on the inside of the second portion of the tip; as these will reduce the internal diameter of the tip, and as the nib will be compressible, this will retain the nib in place during use. Heat staking or gluing of the nib to the tip is also possible.

In one embodiment a liquid may be provided to the nib prior to sample collection in order to dampen the nib. This can facilitate sample collection from a dry sample surface (for example, forensic collection from areas which have been touched; or collection from the anterior nares). In preferred embodiments, the nib can be dry for collection of liquid samples such as blood, saliva, urine, etc.

Suitably, at least one removable cap is provided to shroud the nib before and/or after use. One such cap may be provided over the nib prior to use, and is replaceable after use to protect the sample obtained on the nib.

The nib may be provided with a hydrochromic mark to indicate the quantity of aqueous sample acquired. For example, the mark may be provided at a certain point along the length of the nib, so as to indicate when a predetermined amount of aqueous sample has been drawn into the nib. Alternatively, the mark may take the form of a water-sensitive dye throughout the nib which either appears or disappears in proportion to the amount of water present.

The porous nib in one embodiment may be a sintered porous nib.

The porous nib can comprise porous plastics, porous metals, porous ceramics, and fibers or extruded plastic or metal with internal channels. In a specific embodiment, the porous plastic is a sintered porous plastic. Plastic nibs may be made from a variety of plastics such as polyethylene. Polyethylenes which may be employed include but are not limited to high density polyethylene (HDPE), low density polyethylene (LDPE) and ultra high molecular weight polyethylene (UHMWPE). Nibs may also be made from polypropylene (PP), polyvinylidene fluoride (PVDF), polyamides, polyacrylates, polystyrene, polyacrylic nitrile (PAN), ethylene-vinyl acetate (EVA), polyesters, polycarbonates, or polytetrafluoroethylene (PTFE). Plastic nibs may also be made from more than one of the aforementioned plastics. In one embodiment, a plastic nib is made from about 30% PP and about 70% PE (wt:wt %). In other embodiments when PP and PE are combined, PP may be present in a range of from about 100% to about 0% and PE may be present in a range of from about 0 to about 100% (100% to 0%:0% to 100% wt:wt %). When PE is combined with other polymers, the PE is present in at least about 50% (wt %). In one embodiment the plastic is HDPE. In other embodiments the plastic is UHMWPE, PP, polyamides, or polyacrylic nitrile. Other suitable nib materials include those described in US 2016/0349153.

As described in more detail below, the nib may comprise an active agent for treatment of the sample obtained on the nib, preferably the nib being functionalised with said active agent. The active agent preferably comprises one or more membrane-disrupting reagents that have the ability to lyse cells (eg bacteria) or viruses, releasing components thereof. For example, the DNA and/or RNA of lysed cells may be collected for further processing or analysis; alternatively, lipids, proteins or peptides may be collected, or subcellular organelles, or cellular fragments including cell membrane or cell wall fragments. Preferably the active agent is non-toxic to humans. One preferred active agent comprises a quaternary ammonium compound (QAC), and a more preferable active agent is cetyl pyridinium chloride, although alternatives (for example, as recited in Table 1, either individually or in combination) may be used. In some embodiments the active agent is in lyophilised or dried-down form, and the agent may be re-hydrated by dampening the nib; for example, when the nib is used for collection of a liquid sample such as blood or saliva. Other uses for the active agent may be to capture certain components that may be inhibitory to the analysis process.

As described in more detail herein, we have surprisingly found that certain active agents are not only non-toxic to humans, but are effective at releasing nucleic acids from cells and/or viruses at concentrations which do not interfere with subsequence nucleic acid amplification. This combination of characteristics makes these active agents particularly suitable for the sampling and processing techniques described herein, but also opens the way to a more general use of these agents in preparation of samples for nucleic acid analysis.

In one embodiment, the active agent comprises polyvinylpyrrolidone (PVP; povidone). Polyvinylpyrrolidone (PVP) has previously been used in extraction buffers to aid the removal of phenolic compounds; it is exceptionally good at absorbing polyphenols during DNA purification. Polyphenols are common in many plant tissues and can deactivate proteins if not removed and therefore inhibit many downstream reactions like PCR. In molecular biology, PVP can be used as a blocking agent during Southern blot analysis as a component of Denhardt's buffer. However, we have determined that PVP is able to rupture viruses to release nucleic acids (and/or other viral components), without inhibiting amplification; and so surprisingly we have found that PVP can be dried in to the nib of the collection device described herein.

In one embodiment, the active agent comprises chlorhexidine and/or citral. The nib may be functionalised by drying this agent into the porous matrix; for example, by allowing a solution of chlorhexidine to be absorbed into the nib, and then allowing it to dry before use. Preferably a solution to be dried on to the nib comprises less than or equal to 5%, 4%, 3%, 2.5%, 2%, 1.5%, or 1% active agent. Where multiple active agents are used, this may be a total for all active agents. The % active agent is selected so that when the nib is rehydrated (either from sample, or from addition of a buffer or wash fluid), the final solution contains active agent at less than or equal to 5%, 4%, 3%, 2.5%, 2%, 1.5%, or 1%. For example, this can be achieved by having a nib of known volume which adsorbs a known volume of sample, and can be flushed with a known volume of buffer. In embodiments of the invention, the initial lysis of sample takes place when liquid sample is absorbed into the nib; the lysed sample is then flushed from the nib with a buffer (for example, Tris buffer). This has the effect that the concentration of active agent is higher in the lysis step than in subsequent amplification after washing; thereby permitting the concentration to be optimised for lysis while subsequently being reduced so as not to interfere with amplification.

In a preferred embodiment, the active agent comprises PVP and either or both chlorhexidine and citral. While PVP is particularly useful for releasing nucleic acids from viruses, chlorhexidine and citral are active against microbes such as bacteria; the combination of these active agents provides a broad spectrum release of nucleic acids. In one preferred embodiment, the active agent comprises 0.9% PVP and 0.1% chlorhexidine or citral. Since both chlorhexidine and citral can interfere with amplification at higher concentrations, the initial lysis at higher concentration and wash to reduce concentration described above is more important in this embodiment.

Other active agents may include agents to protect nucleic acids from degradation. For example, alkyl polyglucosides have been shown to bind DNA and may help protect the collected sample against activity of nucleases and increase the storage time of the collected sample; in an embodiment, the agent further comprises an alkyl polyglucoside. PEG and in particular branched PEG (branched, star and comb and to a lesser extend linear PEG) have also been shown to stabilise oligonucleotides through a process of PEGylation. In one embodiment the nib may include a protective agent in addition to a membrane disrupting agent; for example, PEG 6000 at 1% may be dried into the nib in addition to chlorhexidine. This would allow self collection and stabilisation of the collected sample which is then biosafe, for distribution to test facilities without degrading the nucleic acids that are released in making the sample biosafe. PEG 6000 also reduces hydrophobic protein interaction with mucus/fibres and enhances release from the nib.

Although mainly described herein in relation to obtaining and using test samples for analysis of their characteristic nucleic acid content, the present invention can also be applied to immunological (antibody/antigen) testing procedures, or indeed to collection and/or detection of other analytes which may be found in a biological sample where suitable detection reagents exist. For example, lipids, proteins, peptides, subcellular organelles or fragments, cell membrane or cell wall fragments, etc, may all be collected and processed in suitable applications of the invention.

The present invention is able to provide a test sampling device and method for its use, in which the device comprises a porous matrix to absorb test sample in a defined volume, and to expose the sample to an active ingredient that has been pre-functionalised into the nib through a drying process. The sample rehydrates those components as it wicks into the nib. From there, the extracted nucleic acids (or proteins in the case of immunodiagnostics, or lipids, etc) are available immediately, or can dry down during shipping for subsequent elution through buffer exchange by passing buffer back through the nib.

A further aspect of the invention provides the use of PVP in the preparation of nucleic acids for amplification. Preferably this aspect further comprises the use of chlorhexidine and/or citral in the preparation of nucleic acids for amplification.

A still further aspect of the invention provides a method of preparing and amplifying nucleic acids, the method comprising:
combining a sample with a lysis buffer comprising PVP, to release nucleic acids from material in the sample;
subjecting the sample to a nucleic acid amplification process.

Preferably the nucleic acid amplification process is PCR. Preferably the sample is subjected to a nucleic acid amplification process in the presence of PVP. The lysis buffer may also be used as a nucleic acid amplification mix, by inclusion of appropriate reagents (for example, primers, dNTPs, enzymes, etc). These reagents may be present in the lysis buffer in the initial step, or may be added subsequently.

Preferably the lysis buffer further comprises chlorhexidine and/or citral.

PVP may be present in the lysis buffer at less than or equal to 5%, 4%, 3%, 2.5%, 2%, 1.5%, or 1%. In one preferred embodiment, the lysis buffer comprises 0.9% PVP and 0.1% chlorhexidine or citral.

A yet further aspect of the invention provides a nucleic acid amplification mix comprising PVP, and one or more additional reagents for carrying out a nucleic acid amplification process. Preferably the nucleic acid amplification process is PCR.

Preferably the additional reagents are selected from primers, dNTPs, and enzymes. The amplification mix may further comprise chlorhexidine or citral; and more preferably 0.9% PVP and 0.1% chlorhexidine or citral.

Still further aspects of the invention provide a porous nib for collection of biological samples, the porous nib comprising PVP dried therein. The nib may further comprise chlorhexidine or citral dried therein.

### DRAWINGS AND EXEMPLIFICATION

FIG. 1 is a perspective view of a device for sample collection and preparation;

FIG. 2 shows a cross sectional view of the device

FIG. 3 shows results from using the device

FIGS. 4 and 5 show qPCR amplification curves for samples with a number of different lysis agents

FIG. 6 shows results from amplification with a number of different lysis buffers

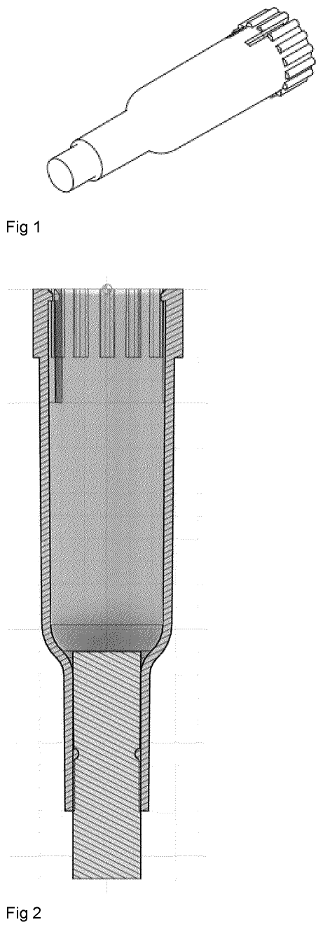

As shown in FIGS. 1 and 2, the device has the general form and size of a conventional pipette tip, comprising a largely cylindrical tip having openings at both ends. A first, larger diameter, section includes a first opening which can engage with a plastic pipette or other device to act as a handle. The second, narrower diameter, section has a second opening within which is received a porous plastic cylindrical nib. In embodiments, the nib may be covered by a removable protective cap (not shown) to protect it; although in other embodiments multiple tip/nib devices could be held in a rack to provide protection when not in use, and to allow for ease of connecting to a pipette. Placing multiple tips/nibs in a rack can also allow for increased throughput by using multiple pipettes or robotic dispensers etc.

The tip is configured so as to provide an interference fit at both openings—clips, ribs, annular rings, detents, or similar may be provided for these purposes. The larger first opening is configured so as to engage with the pipette or other device. The other device may in some embodiments simply be a handle, although it is preferred that the other device allows for ready ejection of the used tip from the device; for example, this may be a lever or push rod or other mechanical means which can engage with the outer rim of the larger opening so as to push it off the other device.

The nib has a sampling end surface which protrudes from the tip, and a wash end surface which is exposed within the tip.

### The Nib

Suitable nib materials include cotton-based materials, or preferably a plastic based matrix such as PE or PVDF which can be manufactured in a range of densities to absorb and retain biological specimens of different viscosities. The precise detail of the nib construction may vary depending on the specific application for use of the invention, and the skilled person will be able to select appropriate materials and densities. For example, where the sample to be collected is saliva, one such suitable material is a cylindrical PE/PP wick of −90% porosity.

Marker pen nibs are often made of porous, pressed fibres such as felt or cellulose, or of porous, pressed plastic spheres, to create an open pore structure. The nib in the present invention may suitably be of similar material or structure, and preferably has a defined porosity (void fraction).

The nib can be designed to be task-specific or designed to cope with a range of different tasks. For forensics work it is convenient to have a nib design that allows fine swabbing of a surface for some applications, or a broad surface for sampling a wider area. It may also be convenient to provide a nib which may be used when damp, to collect dry samples for example from surface swabbing or samples from the anterior nares or the epidermis; or to provide a nib which may be used when dry, to collect liquid samples such as blood, urine, saliva, etc.

Felt pen nibs are often designed to allow ink strokes that are either fine or broad from the same single nib. The same design principles may be employed here in the nib; for example a slanting chisel form of the end surface which enables samples to be taken from a surface as a fine point, a fine edge, or as a broad stroke. However, the nib need not have the structural features described and illustrated there. At its simplest it may present merely a blunt or rounded end.

The porous form of the nib will provide capillary spaces for absorption of liquid test sample. The porous structure can be designed to draw a known volume of sample fluid, e.g. saliva, into its capillary spaces, thereby regulating the amount of sample taken and making the test more consistent.

The nib may have been treated to form a negatively charged surface and/or treated with an anionic detergent and/or treated with a biocidal agent to provide a means of lysing or removing cellular or viral components of the sample from the target nucleic acid components.

Cellulose fibres have a slight negative (anionic) charge. The hydroxyl groups (—OH) of cellulose can also be partially or fully reacted with various reagents to afford derivatives with useful properties. The nib may thus be provided with a negatively charged surface which binds and retains positively charged ions (such as Fe³⁺) from the test sample, and the bulb used to flow neutral or negatively charged ions and molecules (such as DNA) off the nib and away from cationic components.

Most living cells such as bacteria are surrounded by a fatty lipid bilayer. Lipid composition is not the same across subcellular membranes—mammalian plasma membranes having higher cholesterol and sphingolipid content. Viruses also have envelope lipids which are considered to be the same as the host membranes (phospholipids, sphingolipids, some cholesterol).

There are a number of different chemical groups that destroy the lipid layer of cells and consequently display antibacterial or antiviral properties. If these are used to functionalise the nib of the device, then cells (including bacteria) or viruses that soak into the nib will burst, releasing their DNA and RNA (or other cellular or viral components, such as lipids, proteins, subcellular organelles or fragments, membrane or cell wall fragments, etc).

Table 1 below gives examples for functionalising the nib. The mouthwash formulations (shown in the shaded areas) seem particularly useful as they are known to be biosafe and effective, allowing the consumer to place the device into their mouth for self-collection of saliva for testing. For example, cetyl pyridinium chloride (CPC) is the active ingredient of various mouthwashes and has been shown to be a membrane-disrupting agent (Popkin et al, Cetylpyridinium chloride (CPC) exhibits potent, rapid activity against influenza viruses in vitro and in vivo, Pathogens and Immunity, 2017; 2(2):253-69). A particularly preferred formulation is chlorhexidine.

Combinations may provide either targeted activity against the lipid bilayer of different organisms so that the same formulation can be used across a range of targets, or it may provide both a targeted preparation with additional antibacterial or antiviral activity to make the sample biosafe after use.

Reagents may be covalently bound to the nib by suitable chemical linkages through functionalisation (e.g. —OH groups), or they may be dried into the nib material and become active on hydration with the sample.

Drying down the formulation onto the nib minimises dilution of the saliva (or other test sample) and therefore absorption of cleaved material back into the nib matrix.

Mouthwash formulations indicated in Table 1 have been shown to act on viruses and bacteria in a matter of seconds. The nib therefore provides the following support functions:
1. A matrix on which to optionally dry down the antiviral and/or antimicrobial formulation, removing the need to dilute the saliva sample with the treatment;
2. Elicit saliva production when inserted into the mouth;
3. Absorb saliva that is elicited;
4. Contain a known volume of saliva.

A saliva sample may be collected and introduced into a tube for subsequent treatment. Or it may be desired to insert the sampler (nib) in the mouth and collect saliva in situ. In that case, the nib may be treated with compounds that have the ability to break open cells (eg bacteria) or viruses, releasing the DNA and/or RNA and/or other cellular or viral components, but are non-toxic and therefore make the product safe to use in that fashion,

### Using the Device

A particularly preferred application for the device is the collection of saliva samples; for example, for testing for viral infections such as SARS-CoV-2. The use of non-toxic chemicals such as chlorhexidine means that the device can be placed in patients' mouths in order to absorb saliva. In use, an operator may connect the tip to a pipette for ease of handling, and then contact the sampling end with saliva in the mouth. This will then be absorbed into the porous nib, where the chlorhexidine lyses viral capsids if present, to release viral nucleic acids on the nib. The nib at this point will be fully dampened by the saliva.

The operator then removes the pipette with tip and nib from the patient's mouth, and releases the tip from the pipette to allow it to drop into a test tube or other collecting device. At this stage, the tip and nib are in the upright orientation shown in FIG. 2; that is, the larger opening is directed upwards, while the sample surface of the nib is at the lowest point.

The operator may then connect a conventional tip to the pipette, and uses this to dispense a wash buffer into the larger opening of the tip so that the buffer contacts the upper wash surface of the nib. Under gravity, the wash buffer will pass through the nib. This maintains saturation of the nib, but surprisingly and importantly elutes the lysed nucleic acids from the nib into the test tube or other collecting device. It would have been assumed that ordinarily fluid would have to be forced through under pressure, and that gravity would not be sufficient. For example conventional sample preparation often requires centrifugation to release a sample from a capillary matrix. It was completely unexpected that sample would be easily released with such high efficiency from the current device.

Alternatively, rather than using a pipette for both sample collection and manipulation, the tip could be secured to an alternative clip or handle for sample collection, and then transferred into a test tube.

The tip and nib can then be removed from the test tube, and the released sample used for testing.

A particular advantage of the tip and nib devices described herein is their versatility both in terms of the type of sample they can be used for, and of the downstream processing which they are suitable for. In particular, the devices can be used for all types of samples which would normally be collected variously by flocked, cotton, or foam swabs, or cuticle sticks. The tip and nib devices can be used for sample extraction and dilution, are biosafe, and can be used for collection of wet and dry samples, using a single, integrated collection and handling device. This versatility, and comparison with other sample collection techniques, is summarised in the table below.

| | | | | | Integrated | | | |
|---|---|---|---|---|---|---|---|---|
| | | | | | Wet & dry | Biosafe | Extraction | Dilution |
| Swabs | Flocked | Designed for DNA testing | Surface testing, buccal swab, | ✓ | x | x | x | x |
| | | | PCR, molecular testing | | | | | |
| | Cotton | Microbiology, go-to forensics | Surface testing, buccal swab | ✓ | x | x | x | x |
| | | pathologist's tool | | | | | | |
| | Foam | Specimen collection | Saliva, blood or other | ✓ | x | x | x | x |
| | | | physiological specimens | | | | | |
| Sticks | Cuticle | Bevelled edge for collection | Trace DNA evidence such as skin | ✓ | x | x | x | x |
| | sticks | under surfaces, lifting material | cells (e.g. from under the finger nails) | | | | | |
| | | | Tip and nib device | [symbol] | ✓ | ✓ | ✓ | ✓ |

The tip and nib device thus combines multiple functionalities in a single product, reducing the need for use of different sample devices for different types of sample and testing protocols.

### EXAMPLE

FIG. 3 shows results from RT-qPCR analysis of saliva samples spiked with coronavirus 229E from nibs with different functionalisations. Sample was allowed to absorb into the nib, and then Tris buffer pH 7.5 added to the wash surface of the nib so as to release sample from the nib. The released sample was then subjected to RT-qPCR using a conventional protocol.

The vertical axis shows the number of cycles required to detect virus, such that a lower cycle number is more sensitive. From left to right the different functionalised agents on the nibs are: CIT—citral, PP—povidone-iodine, BE—benzalkonium chloride, CE—cetylpyridinium, CH—chlorhexidine diglucorate. The final two plots are control methods of releasing nucleic acids: 95 C—heat to 95 deg C. for 10 minutes, TDI50—proprietary method of killing virus.

### Testing of Lysis Agents

We decided to further investigate the use of different active agents in releasing nucleic acids for detection, with the aim of identifying agents which were non-toxic, effective in releasing nucleic acids from samples, and which did not interfere with amplification.

Initial tests were carried out with cetyl pyridinium chloride (CPC), povidone (PVP), citral, povidone-iodine, and chlorhexidine. Each agent was tested at 2%, 0.6%, 0.2%, and 0.02% concentration; as well as an elution buffer control (Tris-HCl, with no active agent).

An E-RdRP kit from YouSeq was used, which is designed to detect SARS-CoV-2, and includes a positive control containing the SARS-CoV-2 target genes E gene, RdRP gene and human RP (as an internal control). Each reaction included:
10 μL MasterMix
1 μL Primer/Probe
3 μL Water
1 μL Template or Water
5 μL Buffers

PCR was carried out under the following conditions:
55 C for 10 mins RT Step
95 C for 3 mins denaturation
x45 Cycles
95 C for 15 secs
60 C for 60 secs

Amplification was monitored using a Quantstudio Q5.

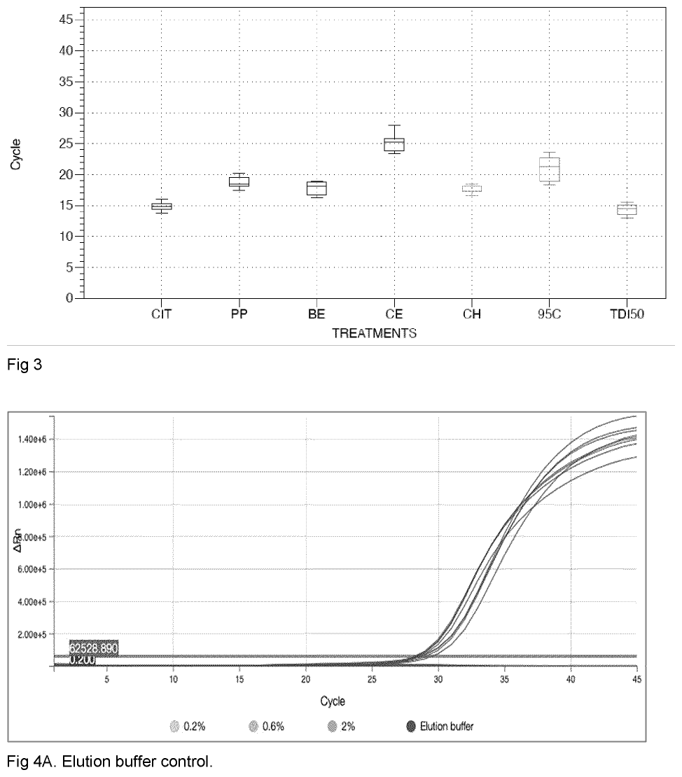

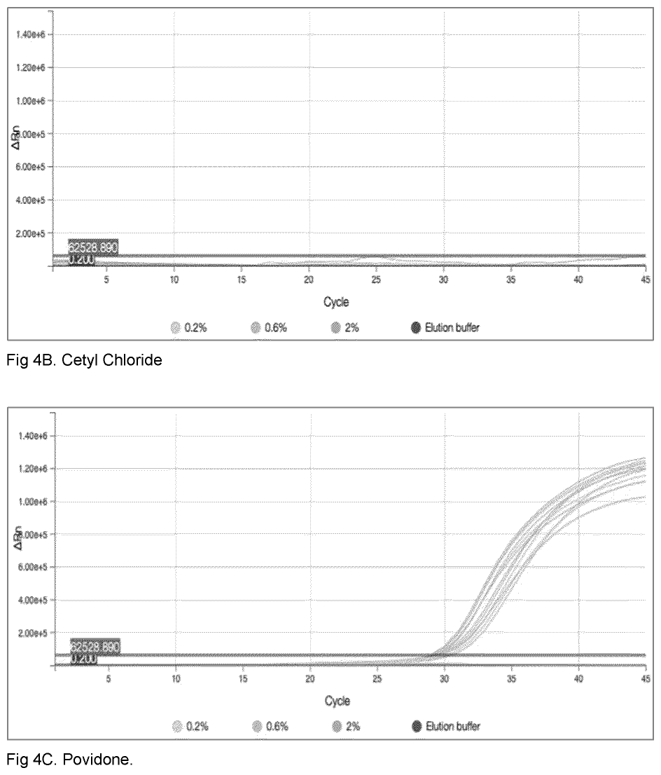

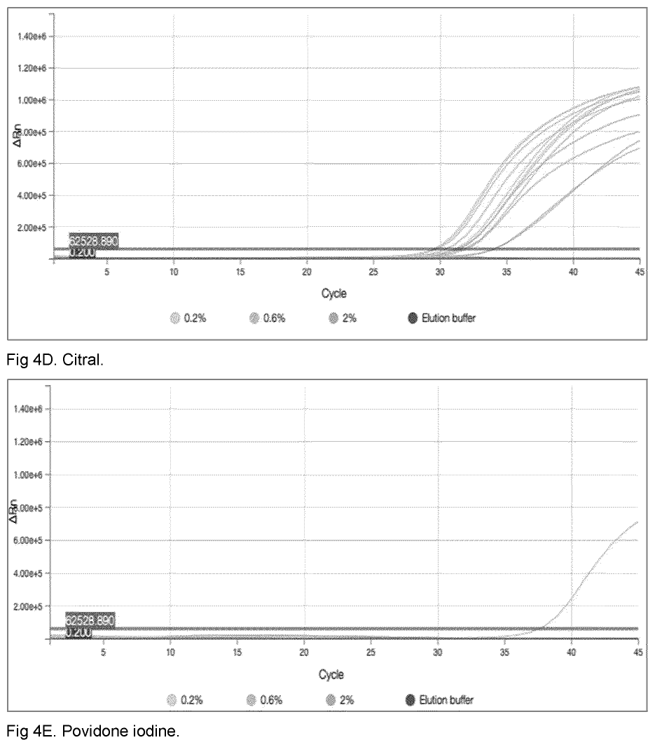

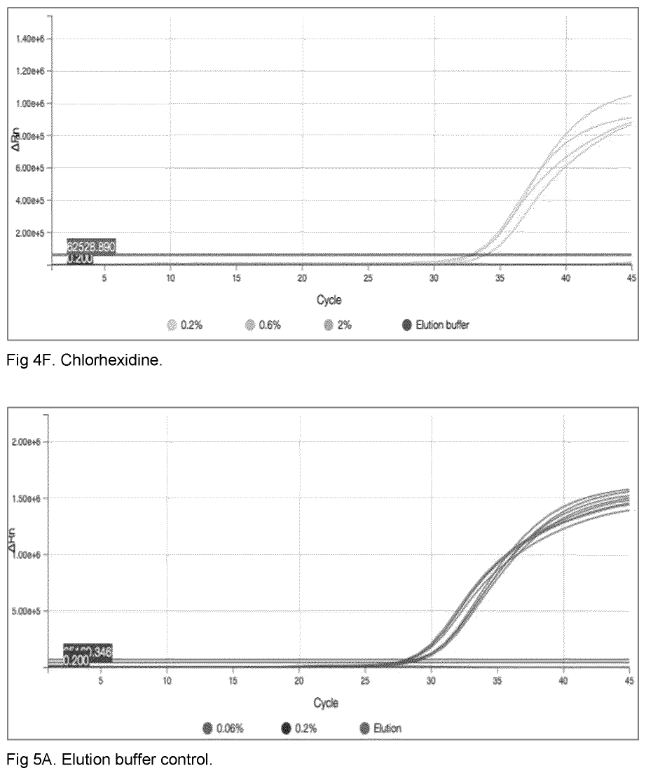

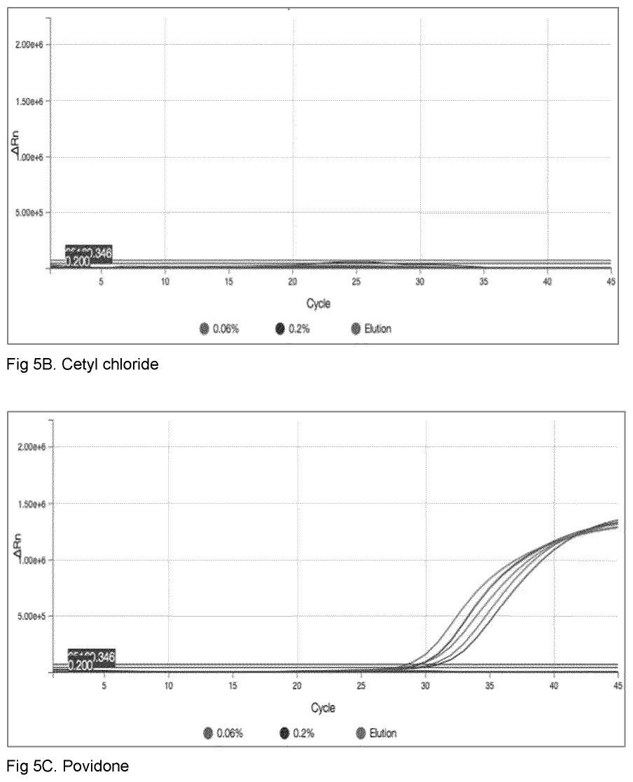

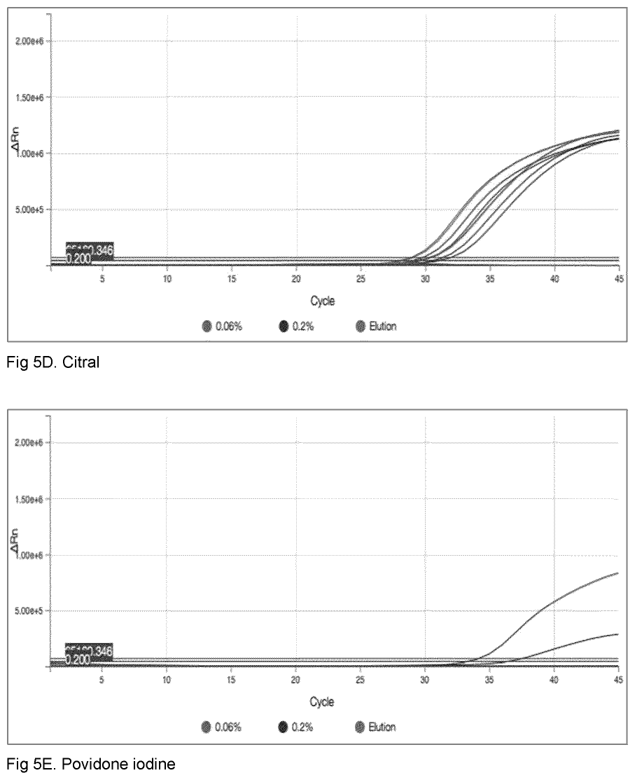

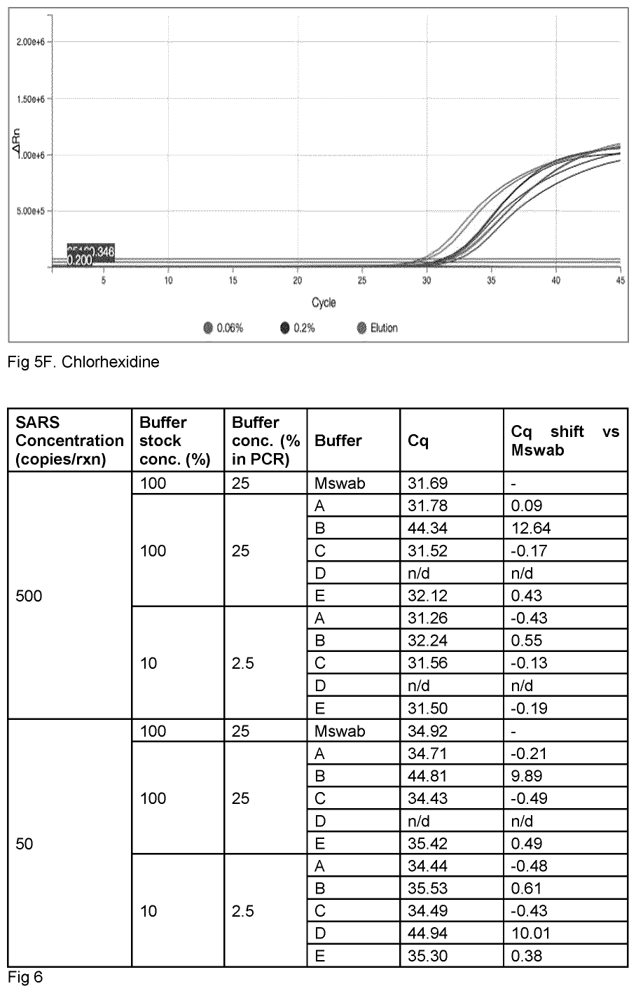

Results are shown in the graphs in FIGS. 4 and 5. It can be seen that CPC and povidone-iodine strongly inhibit amplification; citral and chlorhexidine inhibit at higher concentrations; and povidone does not inhibit amplification. In more detail, the graphs show:

FIG. 4A. Elution buffer control. All show amplifications. This means all controls pass and therefore the data is valid. B. Cetyl chloride. All flat lines, clear PCR inhibition. C. Povidone. All wells show amplification, meaning no significant inhibition. There is no pattern between 2%-0.2%. D. Citral. All show amplifications. However, there is a clear inhibitory effect between 2%-0.2%. E. Povidone iodine. All flat lines (but one weak rep), clear PCR inhibition. F. Chlorhexidine. Only 0.2% wells amplified. A weaker Ct value. Clear PCR inhibitions.

FIG. 5A. Elution buffer control. All show amplifications. This means all controls pass and therefore the data is valid. B. Cetyl chloride. All flat lines, clear PCR inhibition. C. Povidone. All wells show amplification, meaning no significant inhibition. 0.06% does have an improved Ct value compared to 0.2%. D. Citral. All wells show amplification, meaning no significant inhibition. 0.06% does have an improved Ct value compared to 0.2%. E. Povidone iodine. All flat lines (but two weak rep), clear PCR inhibition. F. Chlorhexidine. All wells show amplification, meaning no significant inhibition. 0.06% does have an improved Ct value compared to 0.2%.

The latter three agents were then tested for the ability to release nucleic acids from irradiated whole SARS-CoV-2 particles and then to undergo amplification without inhibition. A control used Mswab buffer, a known buffer suitable for this purpose.

Different combinations of agents were tested:
A=1% Polyvinylpyrrolidone (10,000)
B=0.9% Polyvinylpyrrolidone+0.1% Chlorhexidine
C=0.9% Polyvinylpyrrolidone+0.1% Citral
D=1% Chlorhexidine
E=1% Citral

The results are shown in FIG. 6. It can be seen that all are effective; however, buffers B and C are preferred. Povidone (polyvinylpyrrolidone, PVP) which bears a close physical resemblance to plasma protein is used in the pharmaceutical industry as a synthetic polymer vehicle for dispersing and suspending drugs. It also acts as a disintegrant and tablet binder. It appears as white to off-white hygroscopic powder in its pure form and is readily soluble in water. Povidone itself has no reported microbicidal activity, although povidone-iodine exhibits rapid, potent, broad-spectrum antimicrobial properties. Povidone has been found to have a strong inhibitory action on viruses without appearing significantly toxic to animals. The combination of PVP which has no antimicrobial activity with chlorhexidine and citral which do have antimicrobial activity, makes a broad spectrum disruptor to release nucleic acids for qPCR. This has not been done before, and indeed compounds such as chlorhexidine are generally seen as PCR inhibitors. We have determined that combinations can be used to reduce their impact on qPCR and remain effective as disruptors.

TABLE 1
Ingredient

| | | Active Ingredient | | |
|---|---|---|---|---|
| Group | Active ingredient | concentration | | Reference |
| Monoterpenes | Citral | 25 | μg/ml | Astani et al, Comparative study on the |
| Derived from | Eucalyptus oil | 100 | μg/ml | antiviral activity of selected |
| Essential Oils | Tea tree oil | 7.5 | μg/ml | monoterpenes derived from essential oils. |
| Chlorhexidine | Chlorhexidine | 0.12% | | Phytother Res. 2010; 24(5): 673-679. |
| | | | | Nazzaro et al, Effect of essential oils on |
| | | | | pathogenic bacteria. Pharmaceuticals |
| | | | | (Basel). 2013; 6(12): 1451-1474. |
| Saponins | triterpenoid | | | Simoes et al, Mechanism of antiviral activity of |
| | saponin | | | triterpenoid saponins. Phytother Res. 1999 |
| | | | | June; 13(4): 323-8 |
| | Polyphylla | 6.25-50 | μg/mL | Pu et al, Polyphylla saponin I has antiviral |

| | | | |
|---|---|---|---|
| | saponin I | | activity against influenza A virus. Int J Clin Exp |
| | | | Med. 2015; 8(10): 18963-18971. |
| Povidone- | Povidone-iodine | 0.23%; (PVP-I: 0.47 | Kariwa et al, Inactivation of SARS |
| iodine | | and 0.23% w/v) | coronavirus by means of povidone- |
| | | | iodine, physical conditions and |
| | | | chemical reagents. Dermatology. |
| | | | 2006;212 Suppl 1:119-23 |
| | | | Meister et al, Virucidal Efficacy of Different |
| | | | Oral Rinses Against Severe Acute |
| | | | Respiratory Syndrome Coronavirus 2. J |
| | | | Infect Dis. 2020; 222(8): 1289-1292 |
| | | | Capriotti et al, A Novel Topical 2% |
| | | | Povidone-lodine Solution for the |
| | | | Treatment of Common Warts: A |
| | | | Randomized, Double-Blind, Vehicle- |
| | | | Controlled Trial. Dermatol Ther |
| | | | (Heidelb). 2015; 5(4): 247-252. |
| | | | Nakagawa et al, The efficacy of |
| | | | povidone-iodine products against |
| | | | periodontopathic bacteria. Dermatology. |
| | | | 2006; 212 Suppl 1: 109-11 |
| Quaternary | Cetylpyridinium | CPC is used in | Popkin et al, Cetylpyridinium Chloride (CPC) |
| ammonium | chloride (CPC) | medicated oral | Exhibits Potent, Rapid Activity Against Influenza |
| compounds | | rinses at | Viruses in vitro and in vivo. Pathog Immun. |
| | | concentrations | 2017; 2(2): 252-69 |
| | | 0.025-0.075% w/v | Baker et al, Repurposing Quaternary Ammonium |
| | | (250-750 μg/ml) in | Compounds as Potential Treatments for COVID-19, |
| | | the UK, while | Pharm Res (2020) 37:104 |
| | | lozenges sold in | Schrank et al, Disinfectants, Effective |
| | | some countries | against Severe Acute Respiratory |
| | | contain 1.4-3.0 mg | Syndrome-Coronavirus-2? ACS Infect. Dis. |
| | | of CPC. >10 μg/ml | 2020, 6, 1553-1557 |
| | | shown to be | Kersting et al, Multiplex isothermal solid- |
| | | effective. | phase recombinase polymerase |
| | | | amplification for the specific and fast DNA- |
| | | | based detection of three bacterial |
| | | | pathogens, Microchim Acta (2014) |
| | | | 181: 1715-1723 |
| | | | O'Donnell et al, Potential role of oral rinses |
| | | | targeting the viral lipid envelope in SARS-CoV-2 |
| | | | infection, Function, Volume 1, Issue 1, 2020 |
| | | | WO 2005/046738 A2, Virucidal Activities of |
| | | | Cetylpyridinium Chloride |
| Quaternary | Benzalkonium | | Rabenau et al, Efficacy of various disinfectants |
| ammonium | chloride (BAC) | | against SARS coronavirus. J Hosp Infect. |
| compounds | | | 2005; 61(2): 107-111 |
| (continued) | | | Ansaldi et al, (2004). SARS-CoV, |
| | | | influenza A and syncitial respiratory |
| | | | virus resistance against common |
| | | | disinfectants and ultraviolet |
| | | | irradiation. Journal of Preventive |
| | | | Medicine and Hygiene. 45 |
| Mouth wash | Listerine Cool | | Meister et al, Virucidal Efficacy of Different |
| formulations | Mint Ethanol, | | Oral Rinses Against Severe Acute |
| | essential oils | | Respiratory Syndrome Coronavirus 2. J |
| | Dequonal | | Infect Dis. 2020; 222(8): 1289-1292 |
| | Dequalinium | | |
| | chloride (DAC), | | |
| | Benzalkonium | | |
| | cholride (BAC) | | |
| | Oral B Pro - | | |
| | Cetylpyridinium | | |
| | chloride (CPC) | | |
| | Crest Pro-Health | | |
| | Multi-Protection | | |
| | (C21H38ClN) | | |
| | mouthwash - | | |
| | cetylpyridinium | | |
| | chloride(C21H38ClN) | | |
| | BETADINE | | Martinez Lamas et al, (2020), Is |
| | Gargle and | | povidone-iodine mouthwash effective |
| | Mouth Wash | | against SARS-CoV-2? First in vivo tests. |
| | Povidone-iodine I | | Oral Disease Cimolai, N. (2020). Efficacy |
| | (PVP-I) | | of povidone-iodine to reduce viral load. |
| | | | Oral Diseases, |
| | | | https://doi.org/10.1111/odi.13557. |
| | | | Nakagawa et al, The efficacy of |
| | | | povidone-iodine products against |
| | | | periodontopathic bacteria. |
| | | | Dermatology. 2006; 212 Suppl 1: 109-11 |

1. A device for obtaining and optionally processing biological samples for analysis, comprising:
(a) a hollow tip comprising first and second generally cylindrical sections and having first and second openings at first and second ends of the tip, wherein the second section and second opening have a smaller diameter than the first section and first opening, and wherein the first section and first opening are configured to removably attach to a sampling instrument; and
(b) a porous nib having a working surface at a first end exposed or exposable for acquiring a biological sample, a porous structure suitable for the absorption of biological sample matter thus acquired, and for the passage of liquid through the nib; and a washing surface at a second end for application of a wash fluid to the nib; and
wherein the second section and second opening are configured to removably receive the nib, and wherein the porous nib has a length which is greater than the width.

2. The device of claim 1 wherein the length of the first section of the tip is greater than the width of the same section; and preferably also greater than the length of the nib.

3. The device of claim 1 or 2 wherein the nib is held within the tip by an interference fit.

4. The device of any preceding claim wherein the nib is a sintered porous nib.

5. The device of any preceding claim wherein the nib comprises one or more active agents for treatment of the sample obtained on the nib.

6. The device of claim 5 wherein the one or more active agents is/are in lyophilised or dried-down form.

7. The device of claim 5 or claim 6 wherein the one or more active agents comprise polyvinylpyrrolidone.

8. The device of any of claims 5 to 7 wherein the one or more active agents comprise chlorhexidine and/or citral.

9. The device of any of claims 5 to 8 wherein the one or more active agents comprise an agent which protects nucleic acids from degradation; preferably an alkyl polyglucoside.

10. The device of any of claims 5 to 9 wherein, when wash buffer is applied to the washing surface of the nib, gravity pulls said wash buffer through the nib and displaces processed biological sample from the nib.

11. The device of claim 10 where the processed biological sample comprises nucleic acids, and said released and displaced nucleic acids are directly compatible with molecular diagnostic tests without concentration or purification.

12. The device of claim 10 where the processed biological sample comprises proteins, and said released and displaced proteins are directly compatible with immunodiagnostic tests.

13. The device of any of claims 10, 11, or 12 wherein the application of wash buffer and displacement of biological sample are at low risk of generating aerosols.

14. The device of any preceding claim wherein the nib includes curved or rounded faces at either or both of the working and washing surfaces.

15. The use of polyvinylpyrrolidone in the preparation of nucleic acids for amplification.

16. The use of claim 15, further comprising the use of chlorhexidine and/or citral in the preparation of nucleic acids for amplification.

17. A method of preparing and amplifying nucleic acids, the method comprising:
combining a sample with a lysis buffer comprising polyvinylpyrrolidone, to release nucleic acids from material in the sample;
subjecting the sample to a nucleic acid amplification process.

18. The method of claim 17 wherein the nucleic acid amplification process is PCR.

19. The method of claim 17 or 18 wherein the sample is subjected to a nucleic acid amplification process in the presence of polyvinylpyrrolidone.

20. The method of any of claims 17 to 19 wherein the lysis buffer further comprises appropriate reagents for use as a nucleic acid amplification mix.

21. The method of any of claims 17 to 20 wherein the lysis buffer further comprises chlorhexidine and/or citral.

22. The method of any of claims 17 to 21 wherein polyvinylpyrrolidone is present in the lysis buffer at less than or equal to 5%, 4%, 3%, 2.5%, 2%, 1.5%, or 1%.

23. The method of any of claims 17 to 22 wherein the lysis buffer comprises 0.9% PVP and 0.1% chlorhexidine or citral.

24. A nucleic acid amplification mix comprising polyvinylpyrrolidone, and one or more additional reagents for carrying out a nucleic acid amplification process.

25. The mix of claim 24 further comprising chlorhexidine or citral; and more preferably 0.9% polyvinylpyrrolidone and 0.1% chlorhexidine or citral.

26. A porous nib for collection of biological samples, the porous nib comprising polyvinylpyrrolidone dried therein.

27. The nib of claim 26 further comprising chlorhexidine or citral dried therein.
